(19) United States
(12) Patent Application Publication
(10) Pub. No.: US 2009/0023628 A1
(43) Pub. Date: Jan. 22, 2009

(54) Cosmetic Composition and a Process for Preparing This Cosmetic Composition and a Cosmetic Product

(76) Inventors: Luciana Villa Nova Silva, Sao Paulo (BR); Alexandre Roberto Silva, Jundiai (BR); Thiago Braz, Sao Paulo (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/794,428

(22) PCT Filed: Dec. 28, 2005
(86) PCT No.: PCT/BR05/00262

§ 371 Date: Feb. 27, 2008

(30) Foreign Application Priority Data
Dec. 29, 2004 (BR) PI0405956-5

(51) Int. Cl. A61K 38/02; A61K 31/045; A61K 31/715; A61K 31/355; A61P 17/00; A61K 31/07; A61K 31/4166

(52) U.S. Cl. 514/2; 514/730; 514/390; 514/54; 514/725; 514/458

(57) ABSTRACT

The present invention relates to a cosmetic composition comprising: a wetting system that comprises at least glycerin in an amount ranging from 8.0% to 10% by weight, based on the total weight of the composition; an emollient system that comprises at least cetyl lactate and Shea butter; an emulsifying system that comprises at least steareth-2 and steareth-21; an oiliness adsorbing system that comprises at least nylon 12; and a silicone system that comprises at least cyclomethicone and cyclomethicone and dimethicone crosspolymer. This multifunctional cosmetic composition exhibits rapid absorption, properties of differentiated softness, smoothness and intensive and prolonged hydration when applied to the skin. Further, the present invention relates to a process of preparing the referred-to cosmetic composition and to a cosmetic product that comprises said composition.

This application claims the priority of Brazilian patent case No. PI0405956-5 filed on Dec. 29, 2004 which is hereby incorporated by reference.

### FIELD OF THE INVENTION

The present invention relates to a multifunctional cosmetic composition, which exhibits rapid absorption, softness and smoothness properties and intensive and prolonged moisturizing when applied to the skin.

Further, the present invention relates to a specific process of preparing said cosmetic composition.

### DESCRIPTION OF THE PRIOR ART

By virtue of the physicochemical characteristics, common to most glycols available on the market and several emollient agents that contribute to the stability of the formulations, the cosmetic compositions with high concentration of glycols (glycerin, butyleneglycol, propyleneglycol, among others) and emollients, available on the market today, exhibit various sensorial aspects and post-application effects that are undesirable, namely:
1—high degree of oiliness and stickiness;
2—difficult to spread and slow absorption;
3—increase in the degree of oiliness and brightness of the skin;
4—possible formation of comidones and other adverse reactions such as sensitization and skin irritation.

These aspects and effects are due mainly to:
1—a great influence of the high concentrations of glycols and emollients necessary for obtaining a high level of objective moisturizing (measured by equipment) and subjective moisturizing (sensed on the skin after application of the product);
2—a high degree of stickiness and/or oiliness exhibited by such components;
3—occlusive action caused either by some emollient agents or by some wetting agents (for example, glycols), or by some mixture inherent in these preparations;
4—loss of skin barrier structuring caused by the emollient agents used in large amount, with consequent permeation of preservatives, fragrances, chemical sunscreen and various other components of the preparation, through the layers of the skin, where they are potentially irritant.

The applicant indicates hereinafter some relevant documents of the prior art relating to the matter of the present invention.

Document BR 9914749 discloses topical compositions indicated for improving the appearance and feeling of the skin. These compositions comprise a particulate material from 0.5% to 2.5% and an active that may be a retinoid. In addition to these components, others are cited, which may be added to the composition, such as wetting agents, surfactants and emulsifiers.

Document U.S. Pat. No. 6,361,783 discloses cosmetic compositions that comprise ascorbic acid dissolved in a polyol, preferably glycerin at high concentration. Further, silicones, surfactants and other components may be added. This document foresees a composition comprising 33% glycerin. Therefore, glycerin is used for the purpose of stabilizing the formulation. This large amount is detrimental to the sensorial properties (softness, smoothness and texture) since it is known that glycerin concentrations higher than 15% make the preparation extremely sticky.

Document BR 0204618 describes treatment compositions comprising retinoids. Further, to provide other characteristics, several components are added: silicones as softness providing agents; nylon as optical diffuser and still fatty acid esters. This is a formulation indicated for treating sensitive lips and/or skins, having an anhydrous base comprising the active ingredient retinoxytrimethylsilane.

### SUMMARY OF THE INVENTION

An objective of the present invention is to provide a cosmetic composition comprising:
a wetting system that comprises at least glycerin in an amount ranging from 8.0% to 10% by weight, based on the total weight of the composition;
an emollient system that comprises at least cetyl lactate and Shea butter;
an emulsifying system that comprises at least steareth 2 and steareth 21;
an oiliness adsorbing system that comprises at least nylon 12, and
a silicone system that comprises at least cyclomethicone and cyclomethicone and dimethicone crosspolymer.

Another objective of the present invention is to provide a process of preparing the cosmetic composition in question, which comprises the following steps:

1. Preparation of PHASE A:

a) adding, one by one, the components of the wetting system, at a temperature of about 25° C.;

b) mixing them at a frequency ranging from 20 to 2000 rpm for 5 to 10 minutes;

c) after complete solubilization of all the components, heating this phase up to 75° C.;

2. Preparation of PHASE B

a) solubilizing at least one emulsifying agent and thermostable liquid emollients at a temperature of 75° C., mixing them at a frequency ranging from 100 to 250 rpm;

3. Preparation of PHASE C
a) promoting hot emulsifying at a temperature of 75° C., by adding the phase B to the phase A under constant stirring of from 500 to 2000 rpm;

4. Preparation of PHASE D

Adding to the phase C the components of the oiliness adsorbing system under constant stirring of from 500 to 2000 rpm for 3 to 10 minutes;

5. Preparation of PHASE F

a) homogenizing the components of the silicone system, at a temperature of 25° C. at a frequency of from 500 to 1000 rpm;

b) adding the phase F to the phase C at a temperature of 40° C., mixing at a frequency ranging from 200 rpm to 1200 rpm for about 3 minutes.

A further objective of the present invention is to provide a cosmetic composition obtainable by the process described above.

### DETAILED DESCRIPTION OF THE INVENTION

The cosmetic composition of the present invention is multifunctional and exhibits rapid absorption, differentiated properties of softness and smoothness, and intensive and prolonged moisturizing when applied to the skin.

The cosmetic composition of the present invention comprises associating moisturizing agents (wetting agents and emollients) and specific emulsifying agents to obtain a variety of formulations with various functionalities for skin care and protection. It has been found that each of the components of this base, either alone or in combination, has a quite advantageous purpose for skin care, namely:
the combination of the emulsifiers steareth 2 and steareth 21 forms liquid crystals that constitute an intermediate state between the liquid and solid states, which is called mesophase or mosomorphic state. The formation of these structures can vary depending on the concentration of the emulsifiers used as well as on the temperature and cooling/heating speed employed in the process. The network of liquid crystals formed provides retention of water close to the skin, resulting moisturizing that lasts longer;
the addition of glycerin in an amount ranging from 8% to 10% by weight, based on the total weight of the composition, provides adequate moisturizing to the skin without impairing other properties such as spreadability and stickiness;
the combination of all the systems comprised by the cosmetic composition of the present invention provides optimization of the properties of softness, smoothness and spreadability, besides resulting in an intensive and prolonged moisturizing effect, with an improvement in important attributes such as stickiness, formation of a velvety film, among others. Thus, the cosmetic composition in question provides softness, comfort and well-being during and after application.

The main examples of products that can be prepared from the cosmetic composition of the present invention, which is an emulsion of the oil-in-water type for daily or seasonal use, are:
body moisturizer;
face moisturizers for use by day;
face moisturizers for use by night;
anti-marks/wrinkles preparations for the body;
anti-marks/wrinkles preparations for face to be used by day;
anti-marks/wrinkles preparations for face to be used by night;
body or face cosmetic preparations for children's use;
face cosmetic preparations for use after shaving;
localized-action cosmetic preparations, as for the treatment of marks or rings under the eyes;
body or face sunscreens.

The cosmetic composition of the present invention has a number of advantages and characteristics desired in a cosmetic product for skin care, some of which are listed below:

1. a significant improvement in the sensorial aspects:

a—the oily and/or sticky texture replaced by a silky, soft, smooth and velvet aspect during and after application of the preparation;

b—the difficult spreading and slow absorption give way to easy spreading, high spreadability and absorption in ideal time;

c—the unpleasant and uncomfortable feeling at the moment of application of the preparation and afterwards is replaced by a feeling of well-being and pleasure;

2. elimination of undesirable effects after application, such as high rate of comedone formation and of other irritating reactions on the skin;

3. intensive and lasting moisturizing effect from the moment of application to the skin until after application;

4. the cosmetic composition of the present invention does not provide the negative effects caused by compositions having high concentrations of glycols and of emollient substances on the spreadability and stickiness characteristic of the composition;

5. it does not have occlusive action or significant volatility;

6. it promotes the recovery and/or maintains the normal characteristics of the hydrophilic barrier of the skin, and so it does not induce the loss of the structuring thereof;

7. the cosmetic composition of the present invention comprises a moisturizing system (combination of wetting and emollient components) that, combined with the emulsifying agent, able of promoting the formation of liquid crystals, they are capable of providing a high level of skin moisturizing for a long period of time;

8. moreover, since the cosmetic composition of the present invention comprises compounds that, when combined, form liquid crystals, it exhibits:
high stability;
promotion of lasting moisturizing properties;
control over the release of active principles;
a pleasant feeling upon application;

9. as to the moisturizing properties, it can be stated that the structures observed in the liquid crystals are capable of retaining water in a more effective manner, which causes the minimization its evaporation and thus promoting a substantial and lasting effect;

10. the preparation process of the present invention presents solutions to the drawbacks that are observed today in the preparation of cosmetic compositions that confer a high level of moisturizing available on the market, namely:
the aqueous and oil phase prepared do not form particle substances (solids in the form of crystals) or waxy mass and do not promote polymerization reactions;
maintains the emulsifying power inherent in the emulsifying agents chosen and present in the composition.

### The Cosmetic Composition of the Present Invention

As already said before, the cosmetic composition of the present invention comprises:
a wetting system that comprises at least glycerin in an amount ranging from 8.0% to 10% by weight, based on the total weight of the composition;
an emollient system that comprises at least cetyl lactate and Shea butter;
an emulsifying system that comprises at least steareth 2 and steareth 21;
an oiliness adsorbing system that comprises at least nylon 12, and
a silicone system that comprises at least cyclomethicone and cyclomethicone and dimethicone crosspolymer.

In the table below, the preferred proportions of the components present in the cosmetic composition of the present invention are shown. The combination of these components in amounts corresponding to the proportions defined in the following table provides a significant improvement with respect to the compositions known at present. This improvement is noticed with regard to the sensorial aspects (softness, smoothness, prolonged moisturizing, easy sliding) at the moment of application and also with regard to the aspects related to safety of the use of this composition on the skin (results of all the tests were quite satisfactory, as can be seen later).

| Category | Preferred proportions (parts by weight) |
|---|---|
| Wetting system | 8 to 13 |
| Emollient system | 3.5 to 6.5 |
| Oiliness absorbing system | 2 to 6 |
| Silicone system | 6 to 10 |
| Emulsifying system | 3 to 5 |

### Wetting System

The wetting system is constituted by at least glycerin, preferably bidistilled white glycerin. The amount of this system in the composition ranges from 8.0% to 30.0% by weight, preferably from 10.0% to 15.0% by weight, based on the total weight of the cosmetic composition of the present invention. This system may still receive optional wetting agents, which will be defined later

### Emollient System

The emollient system is constituted by at least cetyl lactate and Shea butter. The amount of this system in the composition ranges from 0.1% to 30.0% by weight, preferably from 0.5% to 15.0% by weight, based on the total weight of the cosmetic composition of the present invention. Like the preceding system, this system may still receive optional emollient agents, which will be defined later.

### Emulsifying System

The emulsifying system is constituted by at least steareth 2 and steareth 21. The amount of this system in the composition ranges from 0.1% to 20.0% by weight, preferably from 0.2% to 10.0% by weight, based on the total weight of the cosmetic composition of the present invention. Like the preceding systems, this system may still receive optional emulsifying agents, which will be defined latte.

### Oiliness Adsorbing System

The oiliness adsorbing system is constituted by at least nylon 21. The amount of this system in the composition ranges from 0.1% to 20.0% by weight, preferably from 1.0% to 10.0% by weight, based on the total weight of the cosmetic composition of the present invention. Like the preceding systems, this system may receive optional oiliness adsorbing agents, which will be defined later.

### Silicon System

The silicone system is constituted by at least cyclomethicone and cyclomethicone and dimethicone crosspolymer. The amount of this system in the composition ranges from 0.5% to 30.0% by weight, preferably from 1.0% to 15.0% by weight, based on the total weight of the cosmetic composition of the present invention. Like the preceding systems, this system may still receive optional silicone agents, which will be defined later.

In addition to the already cited components, the cosmetic composition of the present invention may further comprise agents that have specific functions required for each composition necessary for each situation, such as, chelating agents, thickening agents, pH adjusting agents, preservatives, other wetting agents, conditioning agents, other emollients, optional silicones, optional filmogenic agents, oiliness adsorbing agents, skin toning agents, optical diffusing agents, skin tensing agents, sunscreens and UV-filters or actives such as bacteriostatic, bactericidal or antimicrobial, anti-radical, anti-aging, anti-inflammatory actives, among others. This composition may be called a cosmetic base, since it may receive various optional components in its constitution.

Further, according to the optional components added to the composition, protection against sunrays with a good spreading and without causing an increase in oiliness/stickiness can be obtained.

The optional agents that may be added to the systems comprised by the cosmetic composition of the present invention are listed below.

### Additional Wetting Agent

The function of the wetting agent in the cosmetic composition of the present invention is to promote the retention of water in the user's skin, that is to say, to supply water to the skin and also to prevent loss of water from the skin. The wetting agent further helps in raising the efficacy of the emollient, reduces scaling skin and improves the sensorial property of the skin.

A few examples of optional wetting agents that may be added to the cosmetic composition of the present invention are: alkylene polyols and derivatives thereof, glycerol, ethoxylated glycerol, propoxylated glycerol, sorbitol, hydroxypropyl sorbitol among others, C₃-C₆ diols and triols, Aloe vera extract, butyleneglycol, sugars and starches and derivatives thereof, as for example, alkoxylated glucose, hyaluronic acid, glycolic acid, lactic acid, glycolic acid and salicylic acid, panteol, urea, ethoxylated nonyl phenyl, natural oils such as pine oil, oils and waxes and mixtures thereof.

In the preferred embodiments of the present invention an optional wetting agent is used and comprises at least one glycol selected preferably from propyleneglycol, butyleneglycol or diethyleneglycol and combinations thereof.

### Additional Emollient

The function of the emollients in cosmetic composition is to add or replace natural oils to the skin, trying to keep the integrity of the hydrolipidic mantle of the skin. They can also act as solubilizers of sunscreens.

As additional emollients to be added to the composition of the present invention, conventional lipids may be used such as, for example, oils, waxes and other water-insoluble components and polar lipids, which are the those modified so as to increase their solubility in water by esterification of a lipid to a hydrophilic unit like, for example, hydroxyl groups, carbonyl groups, among others. Some components that may be used as emollients are natural oils derived from plants, esters, silicone oils, polyunsaturated fatty acids, lanoline and derivatives thereof. Some natural oils that may be used are derived from apricot kernels, sesame seeds, soybeans, groundnut, coco-nut, olive, cacao butter, almond, carnauba, cotton seed, rice bran, peach stone, mango stone, jojoba, macadamia, coffee bean, grape seed, pumpkin seed, among others and mixtures thereof.

Some ethers and esters may also be used in the function of emollients, such as carboxylic acid C₉-C₃₀ alkyl ester, C₀-C₆ diol monoesters and C₈-C₃₀ carboxylic acid diesters, C₁₀-C₂₀ alcohol sucrose monoesters and combinations thereof. Examples of these compounds are: dicaprylic ether, isopropyl palmitate, dicaprylyl carbonate, C₁₂-C₁₅ alkyl benzoate, isopropyl isononate, sucrose palmitate, sucrose oleate, isostearyl lactate, glyceryl behenate, triglycerol-4 isostearate, lauryl pirrolidone carboxylic acid, pantenyl triacetate and combinations thereof.

Other fatty alcohols, mono-, di- or triglyceride ethers that have a lipophilic nature such as dicaprylylic ether may be used, in addition to synthetic and natural hydrocarbons, organic carbonates such as dicaprylyl carbonate, some types of silicones like cyclomethicone and mixtures thereof.

In addition, various natural compounds may be used as emollients such as, for example, microcrystalized wax, carnauba wax, cupuassu wax, bee wax, ozokerite wax, among others.

In the preferred embodiments of the present invention, an optional emollient, when present, is constituted by various substances of lipophilic nature and different polarities, such as alcohols and fatty acids, esters, ethers, mono-, di- or tri-glycerides, natural or synthetic hydrocarbons, or organic carbons and combinations thereof, preferably ethers, esters and organic carbonates and more preferably dicaprylic ether.

### Optional Emulsifying Agent

As optional emulsifying agents, anionic emulsifiers, non-ionic emulsifiers and polymeric emulsifiers may be used.

Some examples of emulsifying agents may be added, besides those that make part of the already described emulsifying system, such as: components of several categories of substances such as anionic, cationic, preferably non-ionic emulsifiers, such as propoxylated and/or ethoxylated fatty alcohols, sorbitan esters, methyl glucose, propylglyceryl glucose, fatty acids and glycols, fatty acids and sucrose, ethoxylated and/or non-ethoxylated fatty acids and pentaeritritol, copolymers of ethylene oxide and propylene oxide, alkyl glycosides and polyglycosides, ethoxylated and non-ethoxylated animal and vegetable sterols, preferably ethoxylated stearylic alcohols and ethoxylated esters.

The choice of an optional emulsifying agent should be made very carefully, because it may significantly alter the stability of the formulation and may cause a negative impact on the efficacy and safety of the cosmetic composition.

Preferably, glyceryl stearate is used an optional emulsifying agent, when present.

### Additional Oil Adsorbing Agent

As an agent to modify the sensorial feeling, that is to say, to promote adsorption of oiliness, one may add to the composition of the present invention various compounds in combination with Nylon 12 already present in the oiliness adsorbing system, among which: various categories of substances or mixtures thereof, such as polymetylacrylate, polysaccharides, modified polysaccharides, polyethylene, polymethylmetacrylate, acrylate polymers, salicylicate ester, aluminum silicate, magnesium silicate, calcium silicate, magnesium carbonate, calcium carbonate, magnesium oxide, magnesium hydroxide, titanium dioxide, zinc laurate, zinc myristate, polyacrylamide, cellulose, microcrystalline cellulose, maize starch, rice starch, glyceryl starch, maltodextrin, borates, nitrates such as boron nitrate, silicas such as hydrated silica, talc, nylons, mica such as titanated mica, chlorides such as bismuth oxychloride, modified starch such as octenylsuccinate aluminum starch and derivatives thereof, and mixtures thereof.

### Optional Silicone Agent

Silicone exhibits solvent, emollient and skin-conditioning properties. A few examples of optional silicone agents that may be added to the cosmetic compositions of the present invention are: dimethicones, dimeticonols, phenyl trimethicones, volatile and non-volatile silicone oils and, dimethicone-copolyol.

In addition to the above-mentioned components, the cosmetic composition of the present invention may further comprise compounds that are conventionally used in cosmetic compositions of this type and that will be detailed hereinafter.

### Carrier

The water is the base of several possibilities of cosmetic compositions, acting as a carrier for the other components. The composition of the present invention comprises water, preferably demineralized or distilled water at an adequate percentage (q.s.p.) to reach 100% of the formula, based on the total weight of the present composition. Naturally, other cosmetically acceptable carries may be used in the present invention.

### Chelating Agent

The use of a sequestering agent or chelating agent is due to its exhibiting the property of sequestering ions from the solution; they are capable of sequestering calcium and magnesium from the medium, but preferably they exhibit selectivity to bind to iron, manganese and copper ions. Their function is to control a possible oxidation action that may occur and further to promote stability in storage of the cosmetic compositions of the present invention.

A few examples of chelating agents that may be added to the compositions of the present invention include etidronic acid, nitriloacetic acid, polyaminocarboxylic acid such as etilenediaminotetraacetic acid (EDTA) and salts thereof, pentaacetic etilenetriamine acid, di-succinic ethylenediamine acid, nitriloacetates, hydroxyethylethylene triamines, organic phosphates such as sodium hexametaphosphate, sodium tripolyphosphate, sodium gluconate, stylbene, diglutaric ethylenediamine, EGTA and salts, isomers and derivatives thereof.

In the preferred embodiments of the present invention, one preferably uses as a chelating agent etidronic acid or preferably an acid of the ethylenediaminetetracetic acid group, more preferably ethylenedieminetetraacetic disodic acid, at a concentration ranging from 0.05% to 0.50% by weight, based on the total weight of the composition of the present invention.

### Thickening Agent

The function of the thickening agent in cosmetic compositions is to maintain in suspension other components present therein, besides imparting consistency to them.

A few examples of thickening agents that may be used in the present invention are: natural polymer such as alginic acid and derivatives thereof, cellulose and derivatives thereof, scleroglucanes, or preferably some type of gum such as xanthan gum, tara, guar or Arabic gum, but preferably xanthan gum, and synthetic polymers that can also have the function of a polymeric emulsifier formed by carboxyvinylic polymers and copolymers, acrylates, metacrylates, alkyl acrilates, acrylamides, taurates and/or combinations thereof, preferably polymers and copolymers of acrylates and alkyl acrylates and gums, more preferably crosspolymers of acrylates and C₁₀-C₃₀ alkyl acrylate and xanthan gum, and mixtures thereof.

In the preferred embodiments of the present invention, one preferably uses a thickening system composed by crosspolymers of acrylates and C₁₀-C₃₀ alkyl acrylate and xanthan gum, both in an amount ranging from 0.01% to 5.00% by weight, preferably from about 0.10% to about 1.00% by weight, based on the total weight of the cosmetic composition of the present invention.

### Skin-Conditioning Agents

One may use, as conditioners, skin moisturizing or conditioning agents. A few examples of conditioning agents that may be added to the cosmetic compositions of the present invention are guanidine, urea, glycolic acid and glycolate salts, salicylic acid, polyhydroxy alcohols such as sorbitol, manitol, xylitol, erritritol, glycerol hexanotriol, butanotriol, propylene glycol, butylene glycol, hexylene glycol, polyethylene glycol, sugars such as, for example, melibiose and starches, derivatives of sugar and starch derivatives, fructose, glucosamine, hydluronic acid, alantoin and combinations thereof.

One may also use monoesters and C₁-C₃₀ polyesters of sugars and equivalent substances. These esters have a polyol unit and one or more units of carboxylic acid, as for example, sucrose and polyester.

One may further use, as a conditioning agent, cationic polymers. A few examples of cationic polymers suitable for the compositions of the present invention are cationic guar gums such as hydroxypropyl trimethyl ammonium guar, gum, cationic polysaccharides, cationic homopolymers, copolymers derived from acrylic acid and metacrylic acid, cationic cellulose resins, quaternized hydroxyl ethyl cellulose ethers, cationic copolymers of dimethyldialylammonium chloride and of acrylamide and/or acrylic acid, copolymers of dimethyl aminoethylmetacrylate and acrylamide, copolymers of vinyl pirrolidone/vinyl imidazolium metochloride and imines of polyalkylene and ethoxypolyalkylene, quaternized silicones, acrylic acid terpolymers and methyl acrylate and mixtures thereof.

Other conditioning agents indicated for the present invention are monosaccharides, oligosaccharides and polysaccharides, biopolymers such as cellulose, hemicellulose, starch, polyhydroxyalcanoates of bacterial origin, tannins such as the products of polyphenolic plants, colophonies from tree saps, wood lignin and polyactides made by man, alga alginates and proteins such as casein and soybean, biopolymers of uronic acid, highly sulfated polygalactosides, or biopolymers of uronic acid derivatives such as glucuronic acid, glucuronolactone acid, galacturonic acid, galacturonolactone, hydroxypyruvic acid, hydroxypyruvic acid phosphate, ascorbic acid and isomers thereof, di-hydroxytartaric acid, 2-hydroxy-2-methylbutanoic acid, 1-hydroxy-1-cyclopropane carboxylic acid, 2-hydroxyhexanedial, 5-hydroxylisin acid, 3-hydroxy-2-aminopentanoic acid, 4-hydroxy-2,2-diphenylbutanoic acid, 3-hydroxy-3-methylglutaric acid and 4-hydroxy-3-pentenoic acid and natural salts, alga extracts and combinations thereof.

In the preferred embodiments of the present invention, one uses a skin-conditioning system that comprises mono-, oligo-, and polysaccharides, biopolymers of uranic acid highly sulfated polygalactosides and natural salts and/or combinations thereof, preferably constituted by biosaccharide gum 1, seaweed extracts (Phacophyccae and Rhadophyccae) and sorbitol at concentrations ranging from 0.1 to 30.0% by weight, preferably from 1.0 to 15.0% by weight, based on the total weight of the cosmetic composition of the present invention.

### Antioxidant Agent

Antioxidant agents act in protecting the topical composition and the skin against oxidation actions.

Compounds with antioxidant properties that may be added to the compositions of the present invention are: sulfites, ascorbates, amino acids such as glycin, histidin, tyrosine, triptophan and derivatives thereof, imidazols, urocanic acid and derivatives thereof, peptides such as, for example, D,L-carnosins, D-carnosine, L-carnosine, hydrophilic or lipophilic substances or mixtures thereof, such as butyl hydroxyl toluene, butyl hydroxyl anisol or tetradibutyl pentaeritrityl hydroxyhydrocynnamate, vitamin E and derivatives thereof.

In the preferred embodiments of the cosmetic compositions of the present invention, one uses butyl hydroxyl toluene as an antioxidant agent in an amount ranging from 0.01% to 1.00% by weight, preferably from 0.01% to 0.40% by weight, based on the total weight of the composition.

### Preservative Agent

A preservative agent, as its name indicates, provides preservation of the composition to which it is added, that it to say, it provides effective protection to the composition against attack of microbial agents, prolonging it useful life or shelf life.

There is a wide variety of preservative agents suitable for cosmetic compositions, and all those that exhibit this function may be added to the cosmetic composition of the present invention, either alone or in combination.

A few examples of preservative agents to be added to the composition of the present invention are: mixture of various categories of substances such as parabens, organic acids, imidazolidinyls, diazolidines, isothiazolinones, hydroxymethylglycinates, phenolic alcohols and iodo alkyl carbamates, preferably phenolic alcohols and iodo alkyl carbonates and derivatives thereof and/or combinations thereof.

In the preferred embodiments of the present invention, one uses a preservative system that comprises phenoxyethanol and 3-iodo-2-propinylbutyl carbamate in an amount ranging from 0.01% to 3.00% by weight, preferably from 0.10% to 1.50% by weight, based on the total weight of the cosmetic composition of the present invention.

### Active Ingredient System

The active ingredient system may comprise substances of various categories such as alpha-bisabolol, alantoin, glycirrizinates, natural extracts, protein hydrolysates, peptides and polypeptides, flavonoids, sterols, vegetable oils, ceramides, oligo- and polysaccharides, vitamins A, E, and derivatives thereof, more preferably hydrolyzed rice protein, soybean isoflavones, biosaccharide gums 2 and 3, microencapsulated and pure retinol and tocopherol at concentrations ranging from 0.005% to 20.000% by weight, preferably from 0.1 to 10.0% by weight, based on the total weight of the composition of the present invention.

### Fragrance

It is optional to add to the composition of the present invention perfume or fragrance selected from a variety of possible substances. Preferably, one adds fragrance of the floral fresco family, more preferably SC 3158a-Inovação Mod.® from manufacturer Quest International, predominantly Flora Fresh. The amount of fragrance to be added to the cosmetic composition of the present invention preferably ranges from 0.01% to 6.00%, more preferably from 0.05% to 3.00% by weight, based on the total weight of the composition of the present invention.

### pH Adjusting Agent

In order to obtain a final composition with pH values that are neutral or adequate to the skin, one may add to the compositions of the present invention: inorganic hydroxides such as sodium hydroxide, calcium carbonate, citric acid, phosphoric acid, sodium citrate, succinic acid, potassium acetate, sodium chloride, amines such as tertiary amine, triethanolamine and mixtures thereof.

In the preferred embodiments of the present invention, one uses, as pH adjusting agent, triethanolamine in an amount that varies according to the final pH of the product and with the concentration of some polymeric thickening agents, when present, which require neutralization. The preferable amount ranges from 0.1 to 2.0% by weight, based on the total weight of the composition.

### Sunscreens and Ultraviolet Filters

In order to filter the sunrays, one may add sun protection agents, which may be water-soluble or fat-soluble.

A few examples of filters that absorb ultraviolet rays, which are indicated to be added to the cosmetic composition of the present invention are: components of various categories of ultraviolet filters and the particulate physical, chemical and organic sunscreens used in isolation or in mixtures, such as 1-(4-terc-butylphenyl)-3-(4-methoxyphenyl)propane-1,3-dione; sodium and triethanolamine; 2-ethoxyethyl 4-methoxycinnamate; 2,2′-dihydroxy-4-methoxybenzophenone; triethanolamine salisylate; 2,2′,4,4′, tetrahidroxybenzophenone; 2-ethylhexyl 4-methoxycinnamate; 2-hydroxy-4-methoxybenzophenone (Oxibenzone); 2-hydroxy-4-methoxybenzophenone-5 sulfonic acid and the sodium salt thereof; 4-aminobenzoic PABA acid; homomethyl salicylate; titanium dioxide, ethyl N-ethoxy-4-aminobenzoate, 2-ethylhexyl 4-dimethyl-aminobenzoate; 2-ethylhexyl salicylate; isopenthyl 4-methoxycinnamate; 3-(4′-methylbenzylidene)d-l-camphor; 3-benzylidene camphor; 2,4,6-trianilin-(p-carbo-2′-ethylhexyl-l′-oxy)-1,3,5-triazin octyl; zinc oxide; benzoic acid; bis-ethylexylphenol methoxyphenyl triazin; methylene bis-benzotriazolyl tetramethylbutyl-phenol.

In the preferred embodiments of the present invention, for products of topical application one uses, as a sunscreen system and ultraviolet filter, the mixture of 2-ethylexyl p-methoxycinnamate; bis-ethylexyloxyphenol methoxyphenyl triazin and benzophenone 3 at concentrations that may range from 0.1 to 50.0%% by weight, preferably from 1.0 to 25.0% by weigh, based on the total weight of the composition of the invention.

### Other Optional Components

In order to confer to the cosmetic composition of the present application some desirable characteristic that has not yet been achieved with the existing components, one may add optional components that are compatible with its properties. Some of these compounds that may be added to the composition are:
bacteriostatics, bacterizides or antibicrobicides;
stabilizing agents such as sodium chloride;
dyes;
plant extracts: chamomile, rosemary, thyme, calendula, carrot extract, common juniper extract, gentian extract, cucumber extract;
optical diffusers; and
other cosmetically acceptable components that are compatible with the base composition.

### Process of Preparing the Cosmetic Composition of the Present Invention

The process for preparing the cosmetic composition of the present invention employs hot emulsification. This emulsifying process (which uses a temperature of about 75° C.) is applicable in the present invention because it enables one to obtain final compositions exhibiting special physicochemical properties, as for example:
optimum physicochemical stability; and
capability of forming liquid crystals.

Knowing that the formation of these structures may vary as a function of the type and of the concentration of the emulsifiers used, as well as of the temperature employed in the process (intensity and heating/cooling time), it is important to point out that all the steps described hereinafter should be carried out so that the intensive moisturizing effect can be guaranteed.

The steps of the process of preparing the cosmetic composition of the present invention are presented later. Some parameters used in carrying out said steps are pointed out below:
the stirring of the cosmetic composition in all the steps should be constant;
thus, in all the steps there must be a stirring means, that is, one always uses some kind of mixer;
this mixer may be: stirrer, anchor, scraper, naval, rotor/stator homogenizer, turbine, a combination thereof or still another means that is capable of keeping the cosmetic composition in question under constant stirring. Preferably, one uses the combination of a rotor/stator type homogenizer, a stirrer and a scraper; and
one may possibly use a vacuum system.

The process of preparing the cosmetic composition of the present invention comprises the following steps:

1. Preparation of the PHASE A:

(a) adding one of the components of the wetting system at a temperature of about 25° C.;

(b) mixing them at a frequency ranging from 20 to 2000 rpm for 5 to 10 minutes;

(c) after complete solubilization of all the components, heating this phase up to 75° C.

2. Preparation of the PHASE B

(a) solubilizing at least one emulsifying agent and thermostable liquid emollients at a temperature of 75° C., mixing them at a frequency ranging from 100 to 250 rpm.

3. Preparation of the PHASE C

(a) promoting hot emulsification at a temperature of 75° C., by adding the phase B to the phase A under constant stirring of from 500 to 2000 rpm.

4. Preparation of the PHASE D

(a) adding to the phase C the components of the oiliness adsorbing system under constant stirring of 500 to 2000 rpm for 3 to 10 minutes.

5. Preparation of the PHASE F

(a) homogenizing the components of the silicone system, at a temperature of 25° C. at a frequency of 500 to 1000 rpm;

(b) adding the phase F to the phase C at a temperature of 40° C., mixing at a frequency ranging from 20 rpm to 1200 rpm for about 3 minutes.

Optionally, one may prepare other phases

6. Preparation of PHASE E

(a) adding to the phase C at least one preservative agent at a temperature of 45° C., mixing it at a frequency ranging from 20 rpm to 1200 rpm for 3 minutes.

7. Preparation of PHASE G

(a) adding at least one skin-conditioning agent under constant stirring of 500 to 200 rpm for a period of time ranging from 2 to 12 minutes.

8. Preparation of Optional Phase H.

Other components may be added to the formulation after the hot emulsifying phase, as for example preservatives, active ingredients, pH and viscosity adjusters, among others.

An example of a process of preparing the cosmetic composition of the present invention is described hereinafter.
1. Preparation of Phase A

a) solubilizing the selected chelating agent in water (or the carrier selected for the present composition) at a temperature of 25° C., with the aid of a stirrer at a frequency of 20 rpm, a scraper at a frequency of 25 rpm and a rotor/stator type homogenizer at a frequency of 1200 rpm for 3 minutes;

b) adding the wetting agents one by one at a temperature of 25° C.;

c) mixing them with the aid of a stirrer at a frequency of 20 rpm, a scraper at a frequency of 25 rpm and a rotor/stator type homogenizer at a frequency of 1200 rpm for 2 minutes;

d) dispersing thickening agents at a temperature of 25° C. and mixing them with the aid of a stirrer at a frequency of 20 rpm, a scraper at a frequency of 25 rpm and a rotor/stator type homogenizer at a frequency of 1400 rpm for 5 minutes;

e) when all the components have been completely dispersed, heating the phase A up to a temperature of 75° C.
2. Preparation of the Phase B

a) solubilizing the emulsifying agents, the antioxidant agents, the waxy emollients and thermostable liquid emollients at a temperature of 75° C.;

b) mixing them with the aid of a stirrer at a frequency from 100 to 250 rpm;

c) keeping the mixture at a temperature of 75° C. and the frequency of stirring between 100 and 250 rpm, checking the dissolution of all the components of this phase.
3. Preparation of Phase C:

a) promoting the hot emulsification at a temperature of 75° C. by adding the phase B to the phase A;

b) mixing with the aid of a stirrer at a frequency of 20 rpm, a scraper at a frequency of 25° C. and a rotor/stator type homogenizer at a frequency of 1500 rpm for 2 minutes.
4. Preparation of Phase D

a) adding to the phase C at least one oiliness adsorbing agent, at a temperature of 60° C., mixing with the aid of a stirrer at a frequency of 20 rpm, a scraper at a frequency of 25 rpm and a rotor/stator type homogenizer at a frequency of 1200 rpm for 3 minutes.
5. Preparation of Phase E

a) adding to the phase C at least one preservative agent at a temperature of 45° C., mixing it with the aid of a scraper at a frequency of 20 rpm and a rotor/stator type homogenizer at a frequency of 1200 rpm for 3 minutes.
6. Preparation of Phase F

a) homogenizing the components of the silicone system, at a temperature of 25° C., with the aid of at least one mixer at a frequency of 200 to 400 rpm;

b) adding the phase F to the phase C at a temperature of 40° C., mixing with the aid of a scraper at a frequency of 20 rpm and a rotor/stator type homogenizer at a frequency of 1200 rpm for about 3 minutes.
7. Preparation of the Phase G

a) adding to the phase C at least one skin-conditioning agent, at a temperature of 25° C., mixing it with the aid of a scraper at a frequency of 20 rpm and a rotor/stator homogenizer at a frequency of 1200 rpm for 2 minutes.
8. Preparation of the Phase H

a) adding to the phase C the active principles and aromatic compositions sensitive to the variation in temperature at a temperature of 25° C.;

b) mixing them with the aid of a scraper at a frequency of 20 rpm and a rotor/stator type homogenizer at a frequency of 1200 rpm for a period of time that may range from 4 to 8 minutes;

c) adding active principles in the form of microcapsules at a temperature of 26° C., mixing them with the aid of a scraper at a frequency of 20 rpm and a rotor/stator type homogenizer at a frequency of 1200 rpm for about 3 minutes;

d) neutralizing the pH of the composition by adding a pH adjusting agent until a physiological pH ranging from 4.5 to 6.5 is reached;

e) mixing the composition with the aid of a stirrer at a frequency of 20 rpm, a scraper at a frequency of 25 rpm and a rotor/stator type homogenizer at a frequency of 1400 rpm for about 3 minutes.

### EXAMPLES OF THE COSMETIC COMPOSITION OF THE PRESENT INVENTION

The examples given below are preferred embodiments of the cosmetic compositions of the present invention and should not be taken as being limitations thereof. So, may other variations of composition may be carried out within the protection scope delimited by the accompanying claims.

These compositions were made by using the hot-emulsification process. In this regard, one sets forth the phases in which each of the components has added to the composition.

### Example 1

| Phase | Components | Mass composition (%) |
|---|---|---|
| A | Demineralized water | Qsp 100% |
| A | disodic EDTA | 0.10 |
| A | Bi-distilled white glycerin | 10.00 |
| A | Seaweed extract | 2.00 |
| A | Xanthan gum | 0.20 |
| A | Crosspolymer of acrylate/alkyl acrylate | 0.20 |
| B | Vitamin E acetate | 0.50 |
| B | Dicaprylic ether | 2.00 |
| B | Shea butter | 1.00 |
| B | Cetyl lactate | 1.00 |
| B | Glyceryl stearate | 0.30 |
| B | Steareth 2 | 2.40 |
| B | Steareth 21 | 0.80 |
| B | BHT | 0.05 |
| D | Nylon 12 | 2.00 |
| E | Phenoxyethanol | 0.70 |
| E | 3-iodo-2-propinylbutyl carbamate | 0.20 |
| F | Crosspolymer of cyclomethicone and | 3.00 |
| | dimethicone | |
| F | Cyclomethicone | 3.00 |
| G | Biosaccharide gum 1 | 4.00 |
| H | Inovação Mod. ® frangrance | 0.20 |
| H | Triethanolamine | 0.10 |

### Example 2

### Nutritious Moisturizing Emulsion for Body or Face Use

In order to prepare the is composition, one added glycerin (at high a concentration) and seaweeds extract as components of the wetting system, and added shea butter and cetyl lactate as additional emollients.

| Components | Mass composition (%) |
|---|---|
| Demineralized water | Qsp 100% |
| Disodic EDTA | 0.10 |
| Bi-distilled white glycerin | 10.00 |
| Seaweed extract | 3.00 |
| Xanthan gum | 0.20 |
| Crosspolymer of acrylates/C₁₀-C₃₀ alkyl acrylate | 0.20 |
| Vitamin E acetate | 0.50 |
| Dicaprylic ether | 2.00 |
| Shea butter | 1.50 |
| Cetyl lactate | 1.50 |
| Glyceryl stearate | 0.20 |
| Steareth 2 | 2.10 |
| Steareth 21 | 0.70 |
| BHT | 0.05 |
| Nylon 12 | 3.00 |
| Crosspolymer of cyclomethicone and dimethicone | 4.00 |
| Cyclomethicone | 4.00 |
| Biosaccharide gum 1 | 5.00 |
| 3-iodo-2-propinylbutyl carbamate | 0.20 |
| Phenoxyethanol | 0.70 |
| Inovação Mod. ® fragrance | 0.20 |
| Triethanolamine | 0.10 |

### Example 3

### Nutritious Moisturizing Emulsion with PFS 8 for Body or Face Use

To prepare this composition, one added glycerin (at a high concentration) and seaweed extract as components of the wetting system, the sunscreen system and the active principles lycopene, vitamin E acetate, sunflower-seed extract and OPC glycospheres.

| Components | Mass composition (%) |
|---|---|
| Demineralized water | Qsp 100% |
| Disodic EDTA | 0.10 |
| Bi-distilled white glycerin | 8.00 |
| Seaweed extract | 2.00 |
| Xanthan gum | 0.20 |
| Crosspolymer of acrylates/C₁₀-C₃₀ alkyl acrylate | 0.20 |
| Vitamin E acetate | 0.75 |
| Dicaprylic ether | 2.00 |
| Dicapryl carbonate | 2.00 |
| 2-ethylhexyl p-methoxycinnamate | 2.00 |
| Bis-ethylexyloxyphenol methoxyphenyl triazine | 5.00 |
| Cetyl lactate | 1.00 |
| Glyceryl stearate | 0.30 |
| Steareth 2 | 2.50 |
| Steareth 21 | 1.00 |
| Nylon 12 | 2.00 |
| BHT | 0.05 |
| Crosspolymer of cyclomethicone and dimethicone | 3.00 |
| Cyclomethicone | 3.00 |
| Biosaccharide gum 1 | 4.00 |
| 3-iodo-2-propinylbutyl carbamate | 0.20 |
| Phenoxyethanol | 0.70 |
| Inovação Mod ® fragrance | 0.20 |
| Lycopene extracted from tomato | 0.003 |
| Palmitoyl hydroxypropyl trimonium aminopectin/ | 0.03 |
| glycerin crosspolymer, lecithin, grape-seed | |
| extract | |
| Sunflower seed extract | 0.37 |
| Triethanolamine | 0.10 |

### Example 4

### Nutritious Anti-sign Moisturizing Emulsion for Face Use

To prepare this composition, one added glycerin (at a high concentration) and seaweed extract as components of the wetting system, and the encapsulated pure active principles soy isoflavones, retinol and tocopherol and hydrolyzed rice protein.

| Components | Mass composition (%) |
|---|---|
| Demineralized water | Qsp 100% |
| Disodic EDTA | 0.10 |
| Bi-distilled white glycerin | 10.00 |
| Seaweed extract | 2.00 |
| Xanthan gum | 0.20 |
| Crosspolymer of acrylates/C₁₀-C₃₀ alkyl acrylate | 0.20 |
| Vitamin E acetate | 2.00 |
| Dicaprylic ether | 2.00 |
| Shea butter | 1.00 |
| Glyceryl stearate | 0.30 |
| Steareth 2 | 0.30 |
| Steareth 21 | 0.80 |
| Nylon 12 | 2.00 |
| BHT | 0.05 |
| Crosspolymer of cyclomethicone and dimethicone | 4.00 |
| Cyclomethicone | 4.00 |
| Biosaccharide gum 3 | 1.0 |
| Biosaccharide gum 1 | 4.00 |
| 3-iodo-2-propinyl butyl carbamate | 0.20 |
| Phenoxyethanol | 0.70 |
| Inovação Mod. ® fragrance | 0.20 |
| Soy isoflavones | 0.10 |
| Retinol (Vitamin A) and tocopherol (Vitamin E) | 1.20 |
| talaspheres | |
| Tocopherol (Vitamin E) in talaspheres | 0.06 |
| Hydrolyzed rice protein | 2.00 |
| Triethanolamine | 0.10 |

### Example 5

### After-Shave Nutritious Moisturizing Emulsion

To prepare this composition, one added glycerin (at a high concentration) and seaweed extract as components of the wetting system, cupuassu butter as an additional emollient and the encapsulated pure active principles alpha-bisabolol, retinol and tocopherol.

| Component | Mass composition (%) |
|---|---|
| Demineralized water | Qsp 100% |
| Disodic EDTA | 0.10 |
| Bi-distilled white glycerin | 10.00 |
| Seaweed extract | 2.00 |
| Xanthan gum | 0.20 |
| Crosspolymer of acrylates/C₁₀-C₃₀ | 0.20 |
| alkyl acrylate | |
| Vitamin E acetate | 1.00 |
| Dicaprylic ether | 2.00 |
| Cupuassu butter | 3.00 |
| Cetyl lactate | 1.00 |
| Glyceryl stearate | 0.30 |
| Stearet 2 | 2.50 |
| Stearet 21 | 1.00 |
| Alpha bisabolol | 0.40 |
| BHT | 0.05 |
| Crosspolymer of cyclomethicone and | 4.00 |
| dimethicone | |
| Cyclomethicone | 4.00 |
| Biosaccharide gum 3 | 0.60 |
| Biosaccharide gum 1 | 3.00 |
| 3-iodo-2-propinylbutyl carbamate | 0.20 |
| phenoxyethanol | 0.70 |
| Inovação Mod. ® fragrance | 020 |
| Retinol (vitamin A) tocopherol | 1.20 |
| (vitamin E) talaspheres | |
| Triethanolamine | 0.10 |

To exemplify the process of preparing the cosmetic composition of the present invention, one will use Example 4—Nutritious Anti-sign Moisturizing Emulsion for Face Use already described.
1. Preparation of Phase A:

a) disodic EDTA was solubilized in water at a temperature of 25° C. with the aid of a stirrer at a frequency of 20 rpm, a scraper at a frequency of 25 rpm and a rotor/stator type homogenizer at a frequency of 1200 rpm for 3 minutes;

b) seaweed extract and bi-distilled white glycerin extract was added one by one at a temperature of 25° C., and mixed with the aid of a stirrer at a frequency of 20 rpm, a scraper at a frequency of 25 rpm and a rotor/stator type homogenizer at a frequency of 12000 rpm for 2 minutes;

c) xanthan gum and crosspolymer of acrylates/C₁₀-C₃₀ alkyl acrylate was dispersed at a temperature of 25° C. and mixed with the aid of a stirrer at a frequency of 20 rpm, a scraper at a frequency of 25 rpm and a rotor/stator type homogenizer at a frequency of 1400 rpm for 5 minutes;

d) when all the components were completely dispersed the phase A was heated up to a temperature of 75° C.
2. Preparation of the Phase B

a) steareth-2, steareth-21, glyceryl stearate, shea butter and BHT were solubilized in dicaprylic ether and tocoferyl acetate and mixed with the aid of a stirrer at a frequency ranging from 100 to 250 rpm;

b) the mixture was heated up to a temperature of 75° C., and mixed with the aid of a stirrer at a frequency of 250 rpm.
3. Preparation of the Phase C

a) the hot emulsification was promoted at a temperature of 75° C., by adding the phase B to the phase A, mixed with the aid of a stirrer at a frequency of 20 rpm, a scraper at a frequency of 25 rpm and a rotor/stator homogenizer at a frequency of 1500 rpm for 2 minutes.
4. Preparation of the Phase D

a) Nylon 12 was added to the phase C, at a temperature of 60° C., mixing with the aid of a stirrer at a frequency of 20 rpm, a scraper at a frequency of 25 rpm and a rotor/stator homogenizer at a frequency of 1200 rpm for 3 minutes.
5. Preparation of the Phase E

a) 3-iodo-2-propinylbutyl carbamate and phenoxyethanol were added to the phase C at a temperature of 45° C., mixing with the aid of a scraper at a frequency of 20 rpm and a rotor/stator type homogenizer at a frequency of 1200 rpm for 3 minutes.
6. Preparation of the Phase F

a) crosspolymer of cyclomethicone and dimethicone and the cyclomethicone were homogeneized, at a temperature of 25° C., with the aid of at least one mixer at a frequency of 200 to 400 rpm;

b) the phase F was added to the phase C at a temperature of 40° C., mixing with the aid of a scraper at a frequency of 20 rpm and a rotor/stator type homogenizer at a frequency of 1200 rpm for 3 minutes.
7. Preparation of the Phase G

a) Biosaccharide gum 1 was added to the phase C, at a temperature of 25° C., mixing with the aid of a scraper at a frequency of 20 rpm and a rotor/stator homogenizer at a frequency of 12 rpm for 2 minutes.
8. Preparation of the Phase H

a) Inovação Mod.® fragrance was added to the phase C, the biosaccharide gum 3, the soy isoflavones, and the hydrolyzed rice protein at a temperature of 25° C. and mixed with the aid of a scraper at a frequency of 20 rpm and a rotor/stator type homogenizer at a frequency of 1200 rpm for a period of time ranging from 4 to 8 minutes;

b) retinol (vitamin A) and tocopherol (vitamin E) in talaspheres were added at a temperature of 25° C. and mixed with the aid of a scraper at a frequency of 20 rpm and a rotor/stator type homogenizer at a frequency of 1200 rpm for 3 minutes;

c) the pH of the composition was neutralized by adding triethanolamine until physiological pH was reached, which ranges from 4.5 to 6.5, and mixed the composition with the aid of a stirrer at a frequency of 20 rpm, a scraper at a frequency of 25° C. rpm and a rotor/stator type homogenizer at a frequency of 1400 rpm for 3 minutes.

Tests of the Cosmetic Composition of the Present Invention

The composition used in the tests described hereinafter is the one defined in Example 4—Anti-sign Nutritious Moisturizing Emulsion for Face Use.

One carried out tests for evaluation by the consumer/user and also clinical studies so as to prove the efficacy and safety of the compositions considered object of the present invention. The results achieved are given below.

1. Evaluation of the Cosmetic Composition by the Consumers:

These studies aim at evaluating the acceptance by the consumer of the cosmetic composition of the present invention. These studies have been performed together with the clinical study of safety described hereinafter.

One selected 70 volunteers between 30 and 65 years of age, of phototypes I, II, III and IV (I—always gets burned, never gets tanned; II—always gets burned, minimum tannin; III—gets moderately burned, gets gradually tanned; and IV gets little burned, always gets tanned) for individual evaluation of the product. The volunteers who were pregnant or breast-feeding, had skin diseases, excessively oily skin and/or acne scars were excluded.

The volunteers discussed the feelings, benefits and characteristics desired with application of a cosmetic product like this one. The discussion was supervised by two experts in the area and, right afterwards, a question form for performance evaluation and acceptance of the product was distributed and answered during the period of test.

The conclusions of this study after prolonged use of the compositions under test are described below.

a. texture of the composition: the volunteers concluded that the composition is easy to spread over the skin and considered the consistency and the absorption thereof by the skin excellent; they further informed that the product does not have stickiness after application;
b. softness and smoothness provided by the composition: 90% of the consumers identified these qualities on their skins after use of the composition;
c. ease of application of the product: virtually all the consumers considered the product easy to apply;
d. moisturizing feeling: 92% of the volunteers felt that their skin was moisturized right after the first application and that this pleasant effect remained on the skin for a long time;
e. evaluation of the odor of the composition: excellent acceptance of the type and concentration of the perfume contained in the composition, proven by the non-existence of any kind of characteristic and unpleasant odor;
f. evaluation of the oiliness of the composition: the composition, after a prolonged period of time, did not present any significant alteration in the degree of oiliness of the skin of the users. So, the composition has, as a benefit, the fact that it can be applied onto users having oily, normal and dry skin.

2. Clinical Studies

a. This study aims at evaluating the comedogenicity caused by application of the composition to the skin. By comedogenicity one understands the production of comedones on rabbit's ears or subject's backs. Comedones are popularly known in Portuguese as “cravo” (comedo, blackhead), being constituted by keratin and tallow, forming amorphous forms that fill up the pilosebaceus follicle. The conditions that determine its appearance are the obstruction of the follicle by hyperkeratosis and the increase in the production of tallow by the sebaceous glands.

Examples of comedogenic substances are mineral oils, lanoline, squalene, cocoa butter and oleic acid.

For this study, female and male volunteers of all types of skin and with ages ranging from 18 to 45 years were selected. One used the following material, syringe, non-absorbent cotton fabric, antiallergic impermeable adhesive plaster, sample of the cosmetic composition, cyanoacrylate glue, an ordinary optical microscope and a magnifying glass (20×).

The method used for evaluating the comedogenicity of the cosmetic composition was the occlusive patch test or contact or epicutaneous. One applied 0.2 ml of the cosmetic composition to the subject's back in a mapped area of 8 cm². Then the occlusion of this area was made with non-absorbent cotton fabric and impermeable antiallergic adhesive plaster. A counterlateral region with the same area was also occluded by following the same methodology, without application of the cosmetic composition, to serve as a control. This procedure was repeated 3 times a week for 4 weeks, resulting in 28 days of continuous occlusive exposure.

The laboratory evaluation was effected after this period of 28 days. Follicular biopsies were carried out by applying the cyanoacrylate glue to the points where the cosmetic composition had been deposited and at the control point. The slides containing the material obtained in the biopsy were analyzed and compared under a microscope. The evaluations of the slides were carried out by a trained expert supervised by a dermatologist.

The clinical evaluations were made by a dermatologist in the beginning of the study and immediately after removal of the occlusive patch test every 48 hours. Said evaluations were made with the aid of a magnifying glass with white fluorescent illumination. All the product-application areas and the control area were evaluated.

Result: the tests carried out with respect to the samples of the composition proved the absence of comedogenicity therein.

b. A clinical, open, randomized, controlled study of the potential of irritability, sensitization and photoallergy of the skin

This study aims at evaluating the adverse reactions that may be caused by application of the cosmetic composition to the skin. By adverse reactions one understands any sign or symptom triggered by a topical product used in a correct way. As examples of adverse reactions, one can cite eczematous contact dermatitis, urticaria, acne and spots.

The irritation potential of a product depends on a number of variables: the components of the composition, the concentration of each of the components, absorption thereof by the skin, the amount applied to the skin, the state in which the skin is at the time of application, the manner and the frequency of application of the product to the skin and the cumulative effect inherent in the product.

The patch test is the main tool used in the diagnose of reaction caused by cosmetics and in the research of allergenicity. In the research of allergenicity, the following clinical tests are involved: primary and accumulated dermal irritability, cutaneous sensitization, phototoxicity and photoallergy. These consist of repeated application of the product to the skin and have the function of detecting possible irritations or sensitization induction. It is indicated to carry out use tests after approval of the product in the patch tests. With the use tests, one can evaluate the allergenicity, sensorial characteristics of the products, that is, their performance.

In order to carry out the allergenicity tests, 142 female and male volunteers were selected (133 women and 9 men), of all races, with ages ranging from 18 to 62 years, excluding subjects who had skin diseases, lesions or nerves on the back and were pregnant or breast-feeding.

In order to perform this study, the following material was employed: hypoallergenic adhesive patch for patch test with discs of filter paper of 1.0 cm² duly identified, hypoallergenic semipermeable adhesive plaster for occlusion, saline solution and samples of the cosmetic composition. One applied 0.05 g of the cosmetic composition to each area of 1 cm² of the filter-paper disc, and the saline solution to the control disc. These discs were fixed to the subject's back with the aid of adhesive plaster.

The following clinical researches were also carried out:

I. Research of Primary Irritability

The test method used was the patch test or the epicutaneous test (occlusive patch test). The points of application of the tests were the backs of the volunteers, duly protected. The patch test was removed by the researches after 48 hours of contact with the skin and the reactions were written down, 30 minutes after removal.

II. Research of Accumulated Irritability

The sample was applied always in the same region, on the back, duly protected. The applications were carried out every day, the patch test remaining 72 hours on the weekend for 4 consecutive weeks, in a total of 20 applications. The sample was reapplied to the skin always at the same point and the reactions were written down. After 20 consecutive applications, a 10-day rest period followed, when no patch was applied. After this interval of rest, a simple sample patch was applied to the subject's back, virgin area, that is to say, a point where no patch had been applied. The test was removed by the researchers after 48 hours of contact with the skin and the reactions were written down, 30 minutes after removal.

III. Research of Sensitization

The sample was always applied in the same region of the back, duly protected. The applications were carried out 3 times a week for 3 consecutive weeks, every two days, resulting in a total of 9 applications. The patch test was removed by the researchers 24 hours after application. After a series of 9 consecutive applications, a 10-day rest period followed, when no patch was applied. Then, a simple patch of the sample was applied to the volunteer's back in the virgin area. The patch test was removed by the researchers after 48 hours of contact with the skin and the reactions were written down, 30 minutes after removal.

IV. Research of Phototoxicity and Photoallergy

By phototoxicity one understands the increase of reactivity of the skin to ultraviolet light without an immunological base, and by photoallergy one understands the increase of the reactivity of the skin to ultraviolet light with an immunological base.

The test was carried out as follows: the cosmetic composition was applied to the back of the volunteers at a concentration of 0.05 g/cm², always protected. The applications were made twice a week for 3 weeks, resulting in a total of 6 applications. The patch test was removed by the researchers 24 hours after application, the area being immediately evaluated and irradiated with ultraviolet lamp A and B. The non-irritated areas of the back and the eyes were duly protected against the incidence of light. The sample was always applied at the same place. After 6 consecutive applications and irradiations, a 10-day rest period followed, when no patch and no irradiation was effected. Then, a patch was applied to the back in the virgin area. The tests were removed by the researchers 48 hours after application. After removal, the test areas were irradiated with UVA/UVB lamps. The volunteers were instructed to protect the irritated area against sunshine. Evaluations were made 24 and 48 hours after the last irradiation and written down on a form intended for this purpose.

Result: the tests carried out evidenced the absence of skin sensitization, phototoxicity and photoallergy, and no irritation or sensitization process was detected during the study.

c. Clinical Study of Safety

This study was carried out to determine the prevalence of adverse reactions of the skin and the eyes. Further, one evaluated the alteration of oiliness on the skin with application of the product. It was carried out in conjunction with the study for evaluation of perception of the product by the consumer. 70 female volunteers with ages between 34 and 65 years were selected for individual tests of the product, excluding those who had diseases of the skin, excessively oily skin, acne scars or were pregnant or breast-feeding. The volunteers used the cosmetic composition for 91 days. The clinical evaluations were made no days 1, 7, 14, 28, 63 and 91 of the study period.

Result: this test shown the absence of irritation of the skin and the eyes, and no alteration in the degree of oiliness of the user's skin was found.

d. Study of Prolonged Irritation Kinetics by Corneometry

This study aims at evaluating the moisturizing potential of the cosmetic composition of the present invention. For this study, one used: a corneometer that measures the water contents in the skin, specifying the degree of hydration of the skin surface. Volunteers were selected, who were instructed not to use any cosmetic product on their forearms during the three days preceding the beginning of the test. The product was applied in determined areas of 1 cm² of the forearms in an amount of 2 mg. One of the areas is kept without product. The measurements of the corneometer are made in the beginning of the test and 2, 15, 18 and 24 hours after application of the product to the skin. All the measurements are made in a specific room (hydration room), where the temperature and humidity are kept constant (temperature of 22° C. and maximum relative humidity of 55%).

Result: according to this test, it was possible to prove that the cosmetic composition in question promoted hydration of the skin, evidenced by an increase in the corneometry ion the times 2, 15, 18 and 24 hours, with respect to the control.

1. A cosmetic composition characterized by comprising:
a wetting system that comprises at least glycerin, wherein said glycerin is present in an amount ranging from 8.0% to 10.0% by weight, based on the total weight of the cosmetic composition;
an emollient system that comprises at least cetyl lactate and Shea butter;
an emulsifying system that comprises at least steareth-2, steareth-21;
an oiliness adsorbing system that comprises at least nylon 12; and
a silicone system that comprises at least cyclomethicone and cyclomethicone and dimethicone crosspolymer.

2. A cosmetic composition according to claim 1, characterized in that the wetting system is present in an amount ranging from 8.0% to 30.0% by weight, based on the total weight of the composition.

3. A cosmetic composition according to claim 2, characterized in that the wetting system is present in an amount ranging from 10.0% to 15.0% by weight, based on the total weight of the composition.

4. Cosmetic composition according to claim 1, characterized in that the emollient system is present in an amount raging from 0.1% to 30.0% by weight, based on the total weight of the composition.

5. A cosmetic composition according to claim 4, characterized in that the emollient system is present in amount ranging from 0.5% to 15.0% by weight, based on the total weight of the composition.

6. A cosmetic composition according to claim 1, characterized in that the emollient system comprises dicaprylic ether.

7. A cosmetic composition according to claim 1, characterized in that the emulsifying system is present in an amount ranging from 0.1% to 20.0% by weight, based on the total weight of the composition.

8. A cosmetic composition according to claim 7, characterized in that the emulsifying system is present in an amount ranging from 0.2% to 10.0% by weight, based on the total weight of the composition.

9. A cosmetic composition according to claim 1, characterized in that the emulsifying system comprises glyceryl stearate.

10. A cosmetic composition according to claim 1, characterized in that the oiliness adsorbing system is present in an amount ranging from 0.1% to 20.0% by weight, based on the total weight of the composition.

11. A cosmetic composition according to claim 10, characterized in that the oiliness adsorbing system is present in an amount ranging from 0.2% to 10.0% by weight, based on the total weight of the composition.

12. A cosmetic composition according to claim 1, characterized in that the silicone system is present in an amount ranging from 0.5% to 30.0% by weight, based on the total weight of the composition.

13. A cosmetic composition according to claim 12, characterized in that the silicon system is present in an amount ranging from 1.0% to 15.0% by weight, based on the total weight of the composition.

14. A cosmetic composition according to claim 1, characterized by comprising an active ingredient system in an amount ranging from 0.005% to 20,000% by weight, based on the total weight of the composition.

15. A cosmetic composition according to claim 14, characterized in that the active ingredient system is present in an amount ranging from 0.1% to 10.0% by weight, based on the total weight of the composition.

16. A cosmetic composition according to claim 14, characterized in that the active ingredient system comprises the components selected from alpha-bisabolol, alantoin, glycirrinates, natural extracts, protein hydrolyzates, peptides and polypeptides, flavonoids, sterols, plant oils, ceramides, oligo- and polysaccharides, vitamins A, E and derivatives thereof, and combinations thereof.

17. A cosmetic composition according to claim 16, characterized in that the active ingredient system comprises hydrolyzed rice protein, soy isoflavones, biosaccharide gum 2, biosaccharide gum 3, encapsulated pure retinol and tocopherol.

18. A process of preparing the cosmetic composition as defined in claim 1, characterized by comprising the following steps;
I—Preparation of PHASE A:
a) adding, one by one, the components of the wetting system, at a temperature of about 25° C.;
b) mixing them at a frequency ranging from 20 to 2000 rpm for 5 to 10 minutes;
c) after complete solubilization of all the components, heating this phase up to 75° C.;
II—Preparation of PHASE B:
a) solubilizing at least one emulsifying agent and thermostable liquid emollients at a temperature of 75° C., mixing them at a frequency ranging from 100 to 250 rpm;
III—Preparation of PHASE C:
a) promoting hot emulsification, at a temperature of 75° C., by adding the phase B to the phase A under constant stirring of 500 to 2000 rpm;
IV—Preparation of PHASE D:
a) adding to the phase C the components of the oiliness adsorbing system under constant stirring of 500 to 2000 rpm for 3 to 10 minutes;
V—preparation of PHASE F:
a) harmonizing the components of the silicone system at a temperature of 25° C. at a frequency of 500 to 1000 rpm;
b) adding the phase F to the phase C at a temperature of 40° C., mixing at a frequency ranging from 20 rpm to 1200 rpm for about 3 minutes.

19. A process according to claim 18, characterized by comprising the step of preparing the phase E:
adding to the phase C at least one preservative agent at a temperature of 45° C., mixing it at a frequency ranging from 20 rpm to a frequency of 1200 rpm for 3 minutes.

20. A process according to claim 18, characterized by comprising the step of preparing the phase G:
adding at least one skin-conditioning agent under constant stirring of 500 to 2000 rpm for a period of time ranging from 2 to 12 minutes.

21. A process according to claim 18, characterized by comprising the step of preparing the phase H:
a) adding to the phase C the active principles at a temperature of 25° C.;
b) mixing them at a frequency ranging from 20 rpm to 1200 rpm for a period of time ranging from 4 to 8 minutes;
c) adding active principles in the form of microcapsules at a temperature of 25° C., mixing them at a frequency ranging from 20 rpm to 1200 rpm for about 3 minutes;
d) neutralizing the pH of the composition by adding a pH adjusting agent until the physiological pH is reached, which ranges from 4.5 to 6.5;
e) mixing the composition at a frequency ranging from 20 rpm to 1400 for about 3 minutes.

22. A cosmetic composition characterized by being obtainable by the preparation process as defined in claim 18.

23. A cosmetic product characterized by comprising a cosmetic composition as defined in claim 1.

24. A cosmetic product characterized by comprising a cosmetic composition obtained by the preparation process as defined in claim 18.
